(19) United States
(12) Patent Application Publication
(10) Pub. No.: US 2021/0030633 A1
(43) Pub. Date: Feb. 4, 2021

(54) BRUSH-OFF TYPE TOOTH-ATTACHABLE PATCH

(71) Applicant: LG Household & Health Care Ltd., Seoul (KR)

(72) Inventors: Ji-Young Kim, Daejeon (KR); Jae-Hyun Ahn, Daejeon (KR); Seong-Eun Bang, Daejeon (KR)

(73) Assignee: LG Household & Health Care Ltd., Seoul (KR)

(21) Appl. No.: 16/635,819

(22) PCT Filed: Jul. 31, 2018
(86) PCT No.: PCT/KR2018/008717

§ 371 Date: Jan. 31, 2020

(30) Foreign Application Priority Data
Aug. 3, 2017 (KR) 20-2017-0004098

(51) Int. Cl. A61K 8/02; A61K 8/96; A61Q 11/02

(52) U.S. Cl. CPC A61K 8/0208; A61Q 11/02; A61K 8/96

(57) ABSTRACT

The present disclosure provides a pair of tooth-attachable patches for upper teeth and lower teeth, each of the patches including: a drug layer to be attached to teeth; and a backing layer laminated on the drug layer, wherein the heights of the patches for upper teeth and lower teeth are shorter than the crown lengths of maxillary and mandibular central incisors, respectively. The tooth-attachable patches of the present disclosure are prevented from being in contact with the incisal surface of teeth, thereby reducing foreign body sensation while the patches are attached to the teeth.

### TECHNICAL FIELD

The present application claims priority to Korean Patent Application No. 20-2017-0004098 filed on Aug. 3, 2017 in the Republic of Korea, the disclosures of which are incorporated herein by reference. The present disclosure relates to a tooth-attachable patch, more particularly to a brush-off type tooth-attachable patch with improved durability that can be removed by brushing off.

### BACKGROUND ART

In general, a tooth-attachable patch has a structure including a drug layer containing a drug for tooth whitening, a drug for preventing or improving sensitive teeth, etc. and a backing layer for selectively delivering the drug ingredient toward teeth.

Korean Patent Registration No. 10-0458337 proposes preparation of the backing layer as a water-insoluble layer so that the drug ingredient contained in the patch is selectively delivered only to the teeth. However, the patch including the water-insoluble backing layer is inconvenient because the backing layer remaining after the use of the product should be detached. During the detachment, a residue may remain on the teeth and the patch may be broken due to the tensile force of the patch. To solve this problem, a patch product which is dissolved in the mouth after being attached was developed. However, the product which is removed as it is dissolved gradually in the mouth has the problem that the drug ingredient is not selectively delivered to the teeth and the desired medicinal effect is not achieved due to mixing with saliva. In addition, the product which is removed by gradual dissolution in the mouth has the problem that the drug ingredient is ingested and the drug included in an outer layer is not delivered appropriately to the teeth since the outer layer is dissolved earlier than the portion contacted with the teeth.

In order to solve this problem, a brush-off type patch which is not broken down by saliva or water while the patch is used but can be removed by brushing off after use was developed.

This brush-off patch maintains its shape for a certain period of time and then is removed by brushing off as it is broken into small pieces due to decrease in physical strength with time. For this reason, it has weaker physical strength than the peel-off type patch and is broken or cut easily while being attached to the teeth. This leads to foreign body sensation during use.

### DISCLOSURE

### Technical Problem

In order to solve these problems, the inventors of the present disclosure have studied on the structure of a brush-off type tooth-attachable patch and have developed a brush-off type tooth-attachable patch with an optimal structure.

The present disclosure is directed to providing the problem of foreign body sensation during use of a brush-off type tooth-attachable patch.

### Technical Solution

The present disclosure provides a tooth-attachable patch with an improved ability of maintaining the shape of a tooth-attachable patch, particularly a brush-off type tooth-attachable patch with weak physical strength.

Hereinafter, the present disclosure is described in more detail.

An exemplary embodiment of the present disclosure provides a tooth-attachable patch 1 that can be removed by brushing off.

It is a pair of tooth-attachable patches for upper teeth and lower teeth, each of the patches including: a drug layer 10 to be attached to teeth; and a backing layer 20 laminated on the drug layer.

The patch for upper teeth has a height shorter than the crown length of maxillary central incisors, and has a length not in contact with the incisal surfaces of central incisors.

The patch for lower teeth has a height shorter than the crown length of mandibular central incisors, and has a length not in contact with the incisal surfaces of mandibular central incisors.

As used herein, the term ‘crown’ refers to a portion of a tooth that is not hidden by the gum and is visible to the naked eye. In the related art, the crown is considered differently as a clinical crown and an anatomical crown. However, the crown described herein means the clinical crown and is understood to refer to the portion where the tooth is exposed in the mouth.

FIG. 1 shows a tooth-attachable patch according to an exemplary embodiment of the present disclosure. Referring to FIG. 1, for example, when the tooth-attachable patch has a rectangular shape, a long side may be referred to as a width (m) and a short side may be referred to as a height (h). In FIG. 1, the height is shorter than the crown length of teeth.

FIG. 2 is a photograph showing an arch-shaped tooth-attachable patch according to an exemplary embodiment of the present disclosure. Referring to FIG. 2, when one or more sides of the tooth-attachable patch are curved, the linear distance between the lines extending from two short sides is referred to as a width (m). The height (h) of the patch is defined as the linear distance between a tangent line tangential to the most convex portion of the patch and a straight line which is parallel to the tangent line and connecting the vertices connected to the concave portion of the patch.

The inventors of the present disclosure have discovered for the first time that a ‘brush-off’ type patch, which maintains its shape for a certain period of time, and then breaks down into small pieces and is removed by brushing as the physical strength is decreased with time, has a durability problem while it is attached to teeth, and have completed the present disclosure.

The ‘brush-off patch’ is easily cut off by the incisal surface of the tooth while being attached to teeth (for 10-30 minutes) because its physical strength is weaker than that of the peel-off type patch which requires a process of detachment after a certain period of time, thereby causing foreign body sensation during attachment in the mouth.

Specifically, the brush-off patch may have a fracture strength of 0.05-0.3 kgf, specifically 0.06-0.2 kgf, as measured by an Instron tensile strength tester (Zwick Material Prüfung).

Specifically, a patch for oral use (e.g., a tooth-attachable patch, an attachable toothpaste, etc.) attached to the tooth or the boundary of the tooth and the gum may cause foreign body sensation even if it is thin and flexible. In particular, if it is attached up to the back surface or onto the incisal surface of the tooth, it may cause severe foreign body sensation.

In particular, when the patch has weak physical strength, it may be torn or cut off by the force of occlusion of the incisal surfaces of teeth. The cut pieces move around in the mouth, giving severe discomfort and foreign body sensation. Particularly, a brush-off patch with relatively weak physical strength (which is removed by brushing with a toothbrush after being attached to the tooth or the gap between the tooth and the gum for a certain period of time) has the problem that it is cut easily by the incisal surfaces even with a small force in the mouth. Although a method of changing composition or thickness has been attempted to improve this, the problem could not be solved.

As a result of conducting various studied to solve these problems, it has been found out that the foreign body sensation problem can be solved by preparing the patch with a specific size and the present disclosure has been completed.

The inventors of the present disclosure have found out that, since the central incisors from among the six upper front teeth including central incisor, lateral incisors and canines come into contact first during occlusion, the patch is cut off greatly by the incisal surfaces of the central incisors if it is longer than the central incisors. Therefore, foreign body sensation can be reduced by making the height of the patch shorter than the crown length of the central incisors. Likewise, for the lower teeth, the length of the central incisors has the largest effect on foreign body sensation or discomfort from among central incisors, lateral incisors and canines. Therefore, the height of the patch is made to be shorter than the crown length of the central incisors.

On the other hand, because the upper teeth and the lower teeth have different arch structures, the possibility of reaching the incisal surfaces can be minimized and convenience of use can be improved by providing the patch considering the arch structures of the upper teeth and the lower teeth. Although there is a method of attaching the patch so as to cover the gum in order to avoid contact with the incisal surfaces, if the patch contains an ingredient that irritates the gum, e.g., a tooth whitener containing hydrogen peroxide, the size and shape of the patch may be determined to minimize contact with the gum and avoid reaching the incisal surfaces so as to improve convenience of use and safety.

Specifically, it has been found out that, by avoiding reaching the incisal surfaces of teeth, the durability of the patch can be improved and the convenience of use can also be improved significantly.

The height of the patch may be varied depending on the size of the teeth and purposes.

For example, the patch for upper teeth may be prepared to have a height of shorter than 13 mm, specifically shorter than 12.5 mm, at the central portion of the patch. Specifically, the height of the patch for upper teeth may be 6.5-12 mm.

For example, the patch for lower teeth may be prepared to have a height of shorter than 12 mm, specifically shorter than 11.5 mm. Specifically, the height of the patch for lower teeth may be 5.5-11 mm.

For a peroxide-containing tooth-whitening patch that may cause irritation upon contact with the gum, the patch may have a height of 6.5-10.5 mm and the patch for lower teeth may have a height of 6-10 mm.

When the patch has the height described above, the drug contained in the patch may be delivered advantageously without contact with the incisal surfaces of the teeth. In addition, because the patch is not in contact with the incisal surfaces, the shape of the patch can be maintained as desired and the time for contact with the drug can be ensured enough.

The patch of the present disclosure may further include structures that can help brushing off of the patch.

The structures that can help brushing off of the patch may include a cutting line 40 or a fracture-inducing portion 30.

The cutting line 40 may be understood as a line or space formed by cutting the patch open.

The patch may include at least two cutting lines 40.

Specifically, the cutting line may have a length which is shorter than half of the entire height of the patch. In this case, the patch may be attached on the curve surface of the teeth without unintended cutting.

The fracture-inducing portion 30 may be understood as a structure formed to induce easy cutting or breakdown of the patch into predetermined sizes. That is to say, it may include a structure wherein the patch is cut before being attached to teeth and a structure which allows easy cutting or breakdown of the patch during use although it is not cut before attachment.

The shape of the fracture-inducing portion is not particularly limited. For example, the fracture-inducing portion may have a smaller thickness than other portions of a backing layer 20, so that the backing layer 20 can be fractured easily along the portion. Specifically, it may be in the form of unevenness 31. In another exemplary embodiment, the fracture-inducing portion 30 may be in the form of a bubble 32 such that the backing layer 20 can be fractured easily.

The depth of a concave portion 311 of the unevenness 31 is not particularly limited. For example, the depth may be greater than 0 μm and equal to or smaller than 30 μm, specifically 0.1-25 μm, from a convex portion.

The width of the concave portion may be greater than 0 mm and equal to or smaller than 0.1 mm, specifically 0.01-0.08 mm. In this case, the patch may be broken down easily by brushing off while unwanted cutting can be avoided during use.

In another exemplary embodiment of the present disclosure, several fracture-inducing portions may be formed along the edge of the tooth-attachable patch prepared with a specific size.

This type of patch may avoid unnecessary foreign body sensation during use because the patch is not cut by the incisal surfaces during use and the unnecessary portion is removed in advance.

FIG. 2 shows a patch of the present disclosure having a curved edge. Referring to FIG. 2, the patches for upper teeth and lower teeth may be arch-shaped according to the shape of teeth to avoid contact with the incisal surfaces of the teeth.

As used herein, the term ‘incisal surface’ or ‘incisal edge’ refers to the portion where the teeth facing each other (upper tooth and lower tooth) come in contact when the mouth is shut

As used herein, the term “drug layer” refers to a layer containing a drug ingredient that can achieve the purpose of the tooth-attachable patch. For example, the drug layer of a patch for tooth whitening means a layer which contains a tooth-whitening ingredient (specifically, hydrogen peroxide, sodium peroxide, etc.) and the drug layer of a patch for preventing sensitive teeth means a layer which contains sensitive teeth-preventing ingredient such as potassium nitrate, potassium chloride, etc.

The drug ingredient is not particularly limited as long as it can be delivered through the surface of teeth. All the tooth-whitening ingredients and sensitive teeth-preventing ingredients known in the art may be included in the scope of the present disclosure.

As used herein, the term “backing layer” refers to a layer serving to prevent contact with skin in the mouth other than teeth. It contains both a water-insoluble polymer and a water-soluble polymer.

In an exemplary embodiment of the present disclosure, the backing layer may contain a water-insoluble polymer generally used in an oral film. For example, cellulose acetate phthalate, shellac, polyvinyl acetate, ethyl cellulose (EC), poly(methyl methacrylate), a methacryloylethylbetaine-methacrylate copolymer (Yukaformer; Mitsubishi,), a methacrylic acid copolymer (Eudragit L 100, Eudragit L 12,5, Eudragit L 100-55, Eudragit L 30D-55), an aminoalkyl methacrylate copolymer (Eudragit E 100, Eudragit E 12,5, Eudragit RL 100, Eudragit RL 30D), etc. may be used. In addition, the backing layer of the patch to be attached to teeth or around teeth of the present disclosure may contain a water-soluble polymer. As used herein, the term water-soluble polymer refers to a polymer that can be dissolved, swelled or dispersed into small particles in water. In the present disclosure, the term hydrophilic polymer may be used as the same meaning as the water-soluble polymer.

A water-soluble polymer may be used in the drug layer, too. The water-soluble polymer may include a poly(alkyl vinyl ether)-maleic acid copolymer (PVM-MA copolymer; Gantrez AN 119, AN 139, S-97), polyvinyl alcohol, polyacrylic acid, poloxamer 407 (Pluronic, poly(ethylene oxide)-poly(propylene oxide)-poly(ethylene oxide) triblock copolymer), polyethylene oxide (Polyox), a polyvinylpyrrolidone-vinyl acetate copolymer (PVP-VA copolymer; Luviskol VA, Plasdone S PVP-VA), polyvinylpyrrolidone (PVP; K-15-K-120), polyquaternium-11 (Gafquat 755N), polyquaternium-39 (Merquat plus 3330), carboxypolymethylene (Carbomer, Carbopol), hydroxypropylmethyl cellulose, hydroxyethyl cellulose, hydroxypropyl cellulose, gelatin, sodium alginate, etc. Specifically, hydroxypropylmethyl cellulose, polyvinylpyrrolidone or a mixture thereof may be used.

In an exemplary embodiment of the present disclosure, the water-soluble polymer and the water-insoluble polymer may be contained in the backing layer at a weight ratio of 1:0.1-3, specifically 1:0.5-2.

When the water-soluble polymer and the water-insoluble polymer are contained at the weight ratio mentioned above, the patch can maintain its structure stably and can be broken down well when the patch is removed after use. In addition, the patch having the weight ratio mentioned above is not easily cut by the incisal surfaces of teeth and is broken down easily by brushing off.

The patch may contain a drug, specifically an ingredient for tooth whitening, an ingredient for preventing sensitive teeth or a mixture thereof.

The term “breakdown” means division or split of a large piece into smaller pieces. In the present disclosure, the broken down pieces may have a uniform size or various sizes depending on the force or pressure applied from outside. Any word meaning split into several pieces may be used interchangeably with the term breakdown in the present disclosure.

As used herein, the term “patch” refers to an attachable preparation containing a specific ingredient, which may be attached to the surface of teeth, and the type or structure of the patch is not particularly limited.

The tooth-attachable patch according to the present disclosure may be prepared by the methods described in Korean Patent Registration No. 10-0816250 or U.S. Pat. Nos. 6,689,344, 6,682,721 and 6,780,401.

The ingredients contained in the tooth-attachable patch of the present disclosure that can be removed by brushing off are not limited.

### Advantageous Effects

The present disclosure can improve the durability of a brush-off type tooth-attachable patch.

The present disclosure can solve the durability problem of a brush-off type tooth-attachable patch which has very weak physical strength.

According to the present disclosure, the phenomenon of the patch being cut by the incisal surfaces of teeth can be decreased and foreign body sensation in the mouth can be reduced.

### DESCRIPTION OF DRAWINGS

FIG. 1 schematically shows an exemplary embodiment of the tooth-attachable patch according to the present disclosure. m is a long side of the patch and is referred to as width. h is a short side of the patch and is referred to as height.

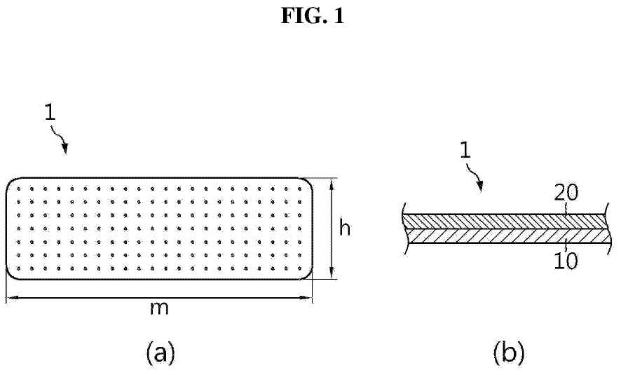

FIG. 2 shows a curved patch according to an exemplary embodiment of the present disclosure.

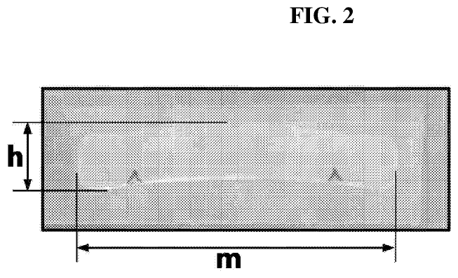

FIG. 3 shows a patch according to an exemplary embodiment of the present disclosure, wherein a cutting line is formed.

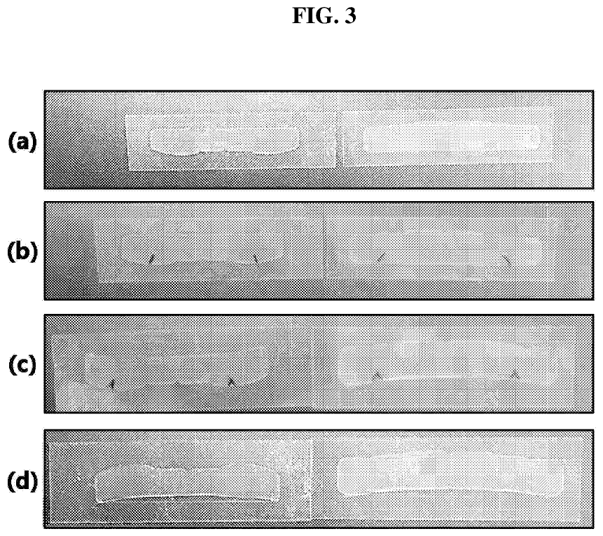

FIG. 4 shows a brush-off type tooth-attachable patch of the present disclosure attached on the surface of teeth.

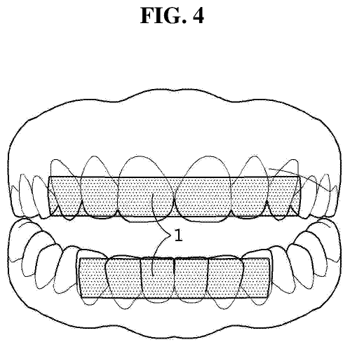

FIG. 5 shows a fracture-inducing portion of a patch according to an exemplary embodiment of the present disclosure, wherein the fracture-inducing portion is unevenness.

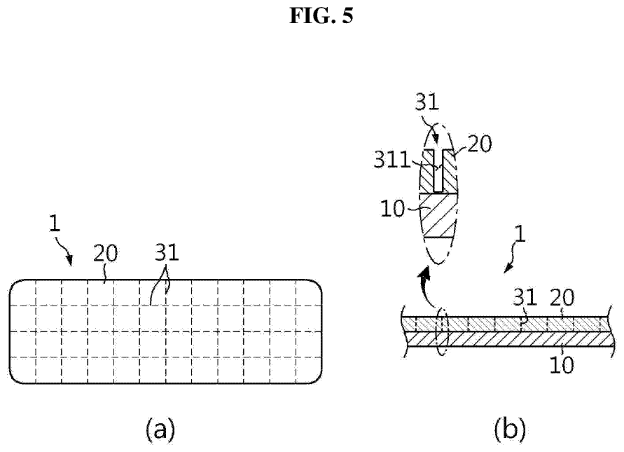

FIG. 6 shows a fracture-inducing portion of a patch according to an exemplary embodiment of the present disclosure, wherein the fracture-inducing portion is a bubble.

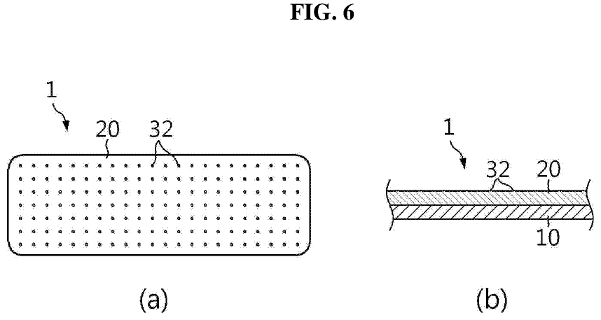

FIG. 7 shows a patch according to an exemplary embodiment of the present disclosure. Several fracture-inducing portions are formed to reflect the curved surface of the patch. Depending on the length of teeth, the unnecessary portion may be removed before use.

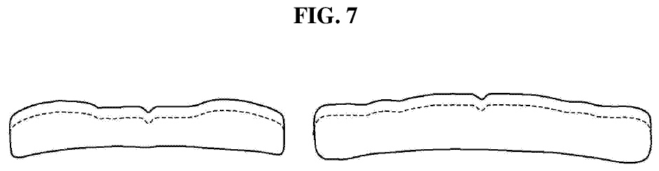

### BEST MODE

Hereinafter, the present disclosure is described in detail through exemplary embodiments. However, the exemplary embodiments according to the present disclosure may be modified into various different forms and the scope of the present disclosure is not limited by the exemplary embodiments. The exemplary embodiments of the present disclosure are provided to more fully explain the present disclosure to those having ordinary knowledge in the art.

1. Preparation of Tooth-Attachable Patch

Patches were prepared according to Table 1 and Table 2. The patches were prepared according to the method of Preparation Example 1 of Korean Patent Registration No. 10-0816250. The prepared patches were cut and processed for application to upper teeth and lower teeth.

(1) Brush-off patch for tooth whitening

TABLE 1

| | | | Comparative | Comparative |
|---|---|---|---|---|
| | Example 1 | Example 2 | Example 1 | Example 2 |
| Drug | Hydrogen | Hydrogen | Hydrogen | Hydrogen |
| layer | peroxide | peroxide | peroxide | peroxide |
| | 2.9% | 2.9% | 2.9% | 2.9% |
| | PVP | PVP | PVP | PVP |
| | 19.0% | 19.0% | 19.0% | 19.0% |
| | Glycerin | Glycerin | Glycerin | Glycerin |
| | 3.0% | 3.0% | 3.0% | 3.0% |
| | Pullulan | Pullulan | Pullulan | Pullulan |
| | 1.0% | 1.0% | 1.0% | 1.0% |
| | Water, etc. | Water, etc. | Water, etc. | Water, etc. |
| | to 100% | to 100% | to 100% | to 100% |
| Backing | EC | EC | EC | EC |
| layer | 10.0% | 10.0% | 10.0% | 10.0% |
| | HPMC | HPMC | HPMC | HPMC |
| | 10.0% | 10.0% | 10.0% | 10.0% |
| | SPAN80 | SPAN80 | SPAN80 | SPAN80 |
| | 5.0% | 5.0% | 5.0% | 5.0% |
| | Castor oil | Castor oil | Castor oil | Castor oil |
| | 5.0% | 5.0% | 5.0% | 5.0% |
| | Ethanol, etc. | Ethanol, etc. | Ethanol, etc. | Ethanol, etc. |
| | to 100% | to 100% | to 100% | to 100% |
| Height, | 10.5 mm | 9.5 mm | 12.0 mm | 15.0 mm |
| for upper | | | | |
| teeth | | | | |
| Height, | 10.0 mm | 9.0 mm | 10.0 mm | 17.0 mm |
| for lower | | | | |
| teeth | | | | |
| Shape | Arched | Rectangular | Arched | Rectangular |

(2) Brush-Off Patch-Type Toothpaste for Preventing and Alleviating Sensitive Teeth

TABLE 2

| | | | Comparative | Comparative |
|---|---|---|---|---|
| | Example 3 | Example 4 | Example 3 | Example 4 |
| Drug | ZnCl₂ | ZnCl₂ | ZnCl₂ | ZnCl₂ |
| layer | 0.09% | 0.09% | 0.09% | 0.09% |
| | CMC | CMC | CMC | CMC |
| | 19.0% | 19.0% | 19.0% | 19.0% |
| | Glycerin | Glycerin | Glycerin | Glycerin |
| | 3.0% | 3.0% | 3.0% | 3.0% |
| | Pullulan | Pullulan | Pullulan | Pullulan |
| | 1.0% | 1.0% | 1.0% | 1.0% |
| | Water, etc. | Water, etc. | Water, etc. | Water, etc. |
| | to 100% | to 100% | to 100% | to 100% |
| Backing | EC | EC | EC | EC |
| layer | 10.0% | 10.0% | 10.0% | 10.0% |
| | Poloxamer | Poloxamer | Poloxamer | Poloxamer |
| | 10.0% | 10.0% | 10.0% | 10.0% |
| | SPAN80 | SPAN80 | SPAN80 | SPAN80 |
| | 5.0% | 5.0% | 5.0% | 5.0% |
| | Castor oil | Castor oil | Castor oil | Castor oil |
| | 5.0% | 5.0% | 5.0% | 5.0% |
| | SLS | SLS | SLS | SLS |
| | 1.0% | 1.0% | 1.0% | 1.0% |
| | Silica | Silica | Silica | Silica |
| | 5.0% | 5.0% | 5.0% | 5.0% |
| | Mint flavor | Mint flavor | Mint flavor | Mint flavor |
| | 0.5% | 0.5% | 0.5% | 0.5% |
| | Ethanol, etc. | Ethanol, etc. | Ethanol, etc. | Ethanol, etc. |
| | to 100% | to 100% | to 100% | to 100% |
| Height, | 11.5 mm | 10.5 mm | 15.0 mm | 15.0 mm |
| for upper | | | | |
| teeth | | | | |
| Height, | 10.5 mm | 10.0 mm | 17.0 mm | 17.0 mm |
| for lower | | | | |
| teeth | | | | |
| Shape | Arched | Rectangular | Arched | Rectangular |

2. How to Use Patch

(1) How to Use a Brush-Off Patch for Tooth Whitening

The brush-off patch for tooth whitening of Table 1 is used as follows. After release paper is removed, the side of the patch to be attached to teeth is attached to the teeth. First, looking into a mirror, the patch for upper teeth is attached so as not to be in contact with the gum. Similarly, the patch for lower teeth is attached so as not to be in contact with the gum. 30 minutes later, the patches are removed by brushing off with a toothbrush soaked with water.

(2) How to Use a Brush-Off Patch-Type Toothpaste for Preventing and Alleviating Sensitive Teeth

The brush-off patch-type toothpaste for preventing and alleviating sensitive teeth of Table 2 is used as follows. After release paper is removed, the patch is attached to teeth. The patch is attached around the site of sensitive teeth, at the boundary between the tooth and the gum. Looking into a mirror, the patch for upper teeth is attached so as not to be in contact with the gum. Similarly, the patch for lower teeth is attached so as not to be in contact with the gum. 10 minutes later, the patches are removed by brushing off with a toothbrush soaked with water.

3. Questionnaire on Feeling after Use

(1) Feeling after Use of Brush-Off Patch for Tooth Whitening

The feeling after use of the tooth whitening brush-off patches of Examples 1-2 and Comparative Examples 1-2 was investigated. 30 participants of different groups were asked to attach the patches of Examples 1-2 or Comparative Examples 1-2 for 30 minutes and to remove them by brushing off.

Then, they were asked to use different products and to answer the questionnaire on feeling after use.

(2) Feeling after Use of Brush-Off Patch-Type Toothpaste for Preventing and Alleviating Sensitive Teeth

The feeling after use of the brush-off patch-type toothpastes for preventing and alleviating sensitive teeth of Examples 3-4 and Comparative Examples 3-4 was investigated in the same manner as in (1). 30 participants of different groups were asked to attach the patches of Examples 3-4 or Comparative Examples 3-4 for 10 minutes and to remove them by brushing off. Then, they were asked to use different products and to answer the questionnaire on feeling after use.

—Scoring criteria—

5: Very comfortable with no foreign body sensation, etc. The patch is not cut easily.

4: Fairly comfortable. The patch remains attached well to teeth surface.

3: Foreign body sensation is felt during use.

2: Foreign body sensation and small pieces are felt in the mouth.

1: Uncomfortable with severe foreign body sensation during use.

4. Questionnaire on Easiness of Removal

The easiness of removal of the patches of Examples 1-2 and Comparative Example 1-2, which require a removal process after use, was investigated. 30 participants of different groups were asked to attach the patches for 10 minutes and to remove them by brushing off. Then, they were asked to use different products and to answer the questionnaire on easiness of removal.

—Scoring Criteria—

5: Very easy to remove with no remains on teeth.

4: Easy to remove, but with a little remains.

3: Not easy to remove and uncomfortable due to a lot of remains.

2: Difficult to remove, and with a lot of remains.

1: Very difficult to remove, and with a great deal of remains.

5. Evaluation Result

The result of the questionnaires on feeling after use and easiness of removal is shown in Table 3.

TABLE 3

| | | | | | Comp. | Comp. | Comp. | Comp. |
|---|---|---|---|---|---|---|---|---|
| | Ex. 1 | Ex. 2 | Ex. 3 | Ex. 4 | Ex. 1 | Ex. 2 | Ex. 3 | Ex. 4 |
| Feeling | 5 | 4 | 5 | 4 | 2.5 | 2 | 2.5 | 2 |
| after use | | | | | | | | |
| Easiness of | 5 | 5 | 5 | 5 | 5 | 5 | 5 | 5 |
| removal | | | | | | | | |

As shown in Table 3, the patches showed significant differences depending on size and shape. That is to say, it was confirmed that even the patches of the same composition can differ in the degree of inducing foreign body sensation in the mouth depending on the height of the patch.

### DESCRIPTION OF MAIN ELEMENTS

1: brush-off type tooth-attachable patch
10: drug layer
20: backing layer
30: fracture-inducing portion
31: unevenness
311: concave portion of unevenness
32: bubble
40: cutting line

### INDUSTRIAL APPLICABILITY

The present disclosure provides a tooth-attachable patch. The patch of the present disclosure maintains its shape well until the patch is removed by brushing off and induces less foreign body sensation.

What is claimed is:

1. A tooth-attachable patch removable by brushing off, wherein
the patch is a pair of tooth-attachable patches for upper teeth and lower teeth,
each of the patches comprises a drug layer to be attached to teeth; and a backing layer laminated on the drug layer, and
the heights of the patches for upper teeth and lower teeth are shorter than the crown lengths of maxillary and mandibular central incisors, respectively.

2. The tooth-attachable patch according to claim 1, wherein the tooth-attachable patch has a physical strength of 0.05-0.3 kgf as measured by an Instron tensile strength tester.

3. The tooth-attachable patch according to claim 1, wherein the patch for upper teeth has a height shorter than 13 mm and the patch for lower teeth has a height shorter than 12 mm.

4. The tooth-attachable patch according to claim 3, wherein the patch for upper teeth has a height of 6.5-12 mm at the central portion.

5. The tooth-attachable patch according to claim 3, wherein the patch for lower teeth has a height of 5.5-11 mm at the central portion.

6. The tooth-attachable patch according to claim 1, wherein the patches for upper teeth and lower teeth further comprise a cutting line or a fracture-inducing portion.

7. The tooth-attachable patch according to claim 1, wherein the patch comprises an ingredient for tooth whitening, an ingredient for preventing sensitive teeth, or a mixture thereof.

8. The tooth-attachable patch according to claim 1, wherein the patch has a curved edge.

9. The tooth-attachable patch according to claim 1, wherein the backing layer of the patch comprises both a water-soluble polymer and a water-insoluble polymer.

10. The tooth-attachable patch according to claim 9, wherein the backing layer of the patch comprises the water-soluble polymer and the water-insoluble polymer at a weight ratio of 1:0.5-2.

11. The tooth-attachable patch according to claim 1, wherein the patch is a brush-off type tooth-attachable patch.
